(19) United States
(12) Patent Application Publication
(10) Pub. No.: US 2026/0193641 A1
(43) Pub. Date: Jul. 9, 2026

(54) GATA2 DEFICIENT CELL LINE AND METHODS OF SCREENING GATA2 VARIANTS

(71) Applicant: Wisconsin Alumni Research Foundation, Madison, WI (US)

(72) Inventors: Emery Bresnick, Middleton, WI (US); Kirby Johnson, Middleton, WI (US)

(73) Assignee: Wisconsin Alumni Research Foundation, Madison, WI (US)

(21) Appl. No.: 19/436,328

(22) Filed: Dec. 30, 2025

Related U.S. Application Data
(60) Provisional application No. 63/742,487, filed on Jan. 7, 2025.

(51) Int. Cl. C12N 15/10; C12N 5/073

(52) U.S. Cl. CPC C12N 15/1068; C12N 5/0605; C12N 2503/00; C12N 2510/00

(57) ABSTRACT

Described herein is a GATA2-deficient human umbilical cord blood-derived erythroid progenitor (HUDEP) cell line, wherein one GATA2 gene copy includes deletion of at least a portion of one or more GATA2 protein-encoding sequences, wherein the deletion results in at least a 25% reduction of GATA2 expression in the GATA2-deficient HUDEP cell line compared to a parent HUDEP cell line without the GATA2 deletion. Also described are methods of screening GATA2 mutations using the GATA2-deficient HUDEP cell lines.

### CROSS-REFERENCE TO RELATED APPLICATIONS

This application claims priority to U.S. Provisional Application 63/742,487 filed on January 7, 2025, which is incorporated herein by reference in its entirety.

### STATEMENT REGARDING FEDERALLY SPONSORED RESEARCH & DEVELOPMENT

This invention was made with government support under DK068634 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on ‎December‎ 12, ‎2025, is named “SEQ_LIST--107668347-P250085US02” and is 31,781 bytes in size. The Sequence Listing does not go beyond the disclosure in the application as filed.

### FIELD OF THE DISCLOSURE

The present disclosure is related to human erythroid GATA2 deficient cell lines and methods of distinguishing GATA2 pathogenic from benign mutants as well as methods of elucidating GATA2 mechanisms in a human hematopoietic progenitor cell.

### BACKGROUND

The GATA2 or GATA-binding factor 2 transcription factor is required for the production and maintenance of hematopoietic stem and progenitor cell (HSPC) populations from which the entire repertoire of adult blood cell types is produced. GATA2 deficiencies in humans result from a reduction in GATA2 level/activity caused by mutations in one copy of GATA2, leading to pathologies including, but not limited to, bone marrow failure and acute myeloid leukemia. A majority of known pathogenic mutations, also termed sequence variants, change the amino acid sequence of GATA2, although not all sequence variation impacts GATA2 activity. GATA2 variants of uncertain significance (VUS) are routinely identified in clinical settings. The pathogenic potential of VUSs may not be predictable from the sequence alone, especially when variants reside in unstructured regions of the protein. In addition, the consequences of GATA2 deficiency on hematopoietic development at the cellular and organismal level have been extensively studied. These studies have enabled the identification of genetic networks controlled by GATA2 that are perturbed by changes to GATA2 levels. It has also been demonstrated that known pathogenic variants, believed to cause loss of function, often retain some GATA2 activities and may even exhibit activities not observed with wild-type GATA2.

Currently, there is not a facile human system to evaluate the activity of GATA2 variants. Described herein is a cell line and cell culture system modeling GATA2 deficiency, based on a human erythroid progenitor cell line, to rapidly assess the activity of GATA2 variants. Curation of GATA2 variants will expedite treatment of patients with GATA2-deficiencies.

### BRIEF SUMMARY

In an aspect, described herein is a GATA2-deficient human umbilical cord blood-derived erythroid progenitor (HUDEP) cell line, wherein one GATA2 gene copy comprises deletion of at least a portion of one or more GATA2 protein-encoding sequences, wherein the deletion results in at least a 25% reduction of GATA2 expression in the GATA2-deficient HUDEP cell line compared to a parent HUDEP cell line without the GATA2 deletion.

In another aspect, a method of screening a GATA2 mutation comprises expressing a variant GATA2 comprising the GATA2 mutation in the GATA2-deficient HUDEP cell line described above, and determining if the variant GATA2 in the GATA2-deficient HUDEP cell line is pathogenic, likely pathogenic, or neutral.

### BRIEF DESCRIPTION OF THE DRAWINGS

FIGS. 1A-G show GATA2 deficiency disrupts the transcriptome of HUDEP-2 cells. (1A) CRISPR targeting strategy for GATA2 knockout. A pair of sgRNAs were employed to remove zinc-finger encoding exons and intervening intronic sequence containing the +9.5 enhancer. (1B) Deletion of zinc fingers and intervening sequence (SEGRECVNCGATATPLWRRDGTGHYLCNACGLYHK; SEQ ID NO:2, zinc finger 1; RRALLVAALLAAGTCCANCQTTTTTLWRRNANGDPVCNACGLYYK; SEQ ID NO: 3, zinc finger 2) to provide GATA ΔZF (SEGRECVNLWRRNANGDPVCNACGLYYK SEQ ID NO: 1) from one allele of wild-type GATA2 resulting from CRISPR targeting. The other allele was unaffected. (1C) Consequence of CRISPR targeting on GATA2 levels. Left, representative Western blot, with β-Actin as a control, demonstrating that HUG2low cells express less GATA2 protein compared to wildtype HUDEP2 cells (compare lane 2 to lane 1). This deficiency can be rescued by lentiviral expression of HA-tagged GATA2 (lane 3). Right, quantitation of GATA2 levels normalized to β-Actin from 3 independent replicates. (1D) Gene ontology analysis for all differentially expressed genes (DEGs) in comparison of HUDEP-2 and HUG2low cells. (1E) Heatmap depicting all DEGs in a comparison of wild type HUDEP cells to HUG2low cells or comparing HUG2low cells expressing HA-GATA2 or empty vector (EV). Four replicates were analyzed for each condition. Roman numerals depict categories of unique and shared genes as indicated by the Venn diagrams in 1F. (1F) Comparison of upregulated and downregulated genes. (1G) The magnitude of differential expression of the 107 GATA2 activated and 38 repressed genes.

FIGS. 2A-C show T354M pathogenic variant disrupts GATA2 function in HUDEP-2 cells. GATA2, wildtype and variants, were expressed in HUG2low cells (GATA2+/-) via lentiviral infection to restore GATA2 to near normal levels. (2A) Left, representative Western blot of GATA2 expression (wildtype and variant) with β-Actin as a control. Right, densitometric analysis of band intensities (n =4 biological replicates). EV, empty vector; WT, wild type; CA, C295A; TM, T354M; Dbl, C295A,T354M double mutant. (2B) Incomplete rescue of GATA2 target gene expression by the T354M variant. 3-4 replicates were analyzed for each condition. (2C) Representative Cut & Tag data at select GATA2 activated genes. The locations of GATA2 occupancy are boxed. Prior to processing, cells were infected with either EV, wild-type GATA2 (G2) or the T354M variant (TM). Isotype-matched IgG was used as a negative control for the GATA2 antibody used for this assay.

The above-described and other features will be appreciated and understood by those skilled in the art from the following detailed description, drawings, and appended claims.

### DETAILED DESCRIPTION

Immortalized erythroid cell lines are important research tools enabling manipulation of molecular targets in the study of erythropoiesis in health and disease. As used herein, human umbilical cord blood-derived erythroid progenitor (HUDEP) cell lines are immortalized human erythroid progenitor cell lines. HUDEP cells are described in Kurita et al. (Kurita R, Suda N, Sudo K, Miharada K, Hiroyama T, Miyoshi H, Tani K, Nakamura Y: Establishment of immortalized human erythroid progenitor cell lines able to produce enucleated red blood cells. PLoS One 2013, 8:e59890.).

HUDEP-2 cells are CD34+ immortalized, but largely normal, human red blood cell progenitors that can be maintained indefinitely in culture and readily differentiated into mature red blood cells. HUDEP-2 cells express CD71, glycophorin A, CD36 and c-Kit. HUDEP-2 cells also express the transcription factor GATA2 and other proteins characteristic of immature hematopoietic progenitor cells. GATA2 deficiency in humans can lead to collapse of the hematopoietic system (bone marrow failure) and/or acute myeloid leukemia (AML).

As described herein, to model GATA2 deficiency, a HUDEP-2 cell line has been generated in which one copy of the GATA2 gene has been deactivated through a CRISPRCas9 targeted deletion. This cell line, termed HUDEP-2 GATA2 low (HUG2low), exhibits an approximately 60% reduction in GATA2 protein expression leading to a substantial disruption in the transcriptome (amalgamated pattern of gene expression) of these cells. Restoring GATA2 to near normal levels in these cells reverses the aberrant pattern of gene expression.

In an aspect, described herein is a GATA2-deficient HUDEP cell line, wherein one GATA2 gene copy comprises a deletion of at least a portion of one or more GATA2 protein-encoding sequences, wherein the deletion results in at least a 20, 25, 30, 35, 40, 45, 50, 55, or 60% reduction of GATA2 expression in the GATA2-deficient HUDEP cell line compared to a parent HUDEP cell line without the GATA2 deletion. GATA2 protein levels can be determined by Western blot analysis as described herein. The second GATA2 gene copy is full-length GATA2.

In an aspect, the HUDEP cell-line is a HUDEP-1, HUDEP-2 or HUDEP-3 cell line, specifically a HUDEP-2 cell line.

Human GATA2 has the canonical amino acid sequence of SEQ ID NO: 4(Uniprot P23769-1). The zinc finger sequences are underlined.

MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG (SEQ ID NO: 4)

Genbank entry NG_029334.1 provides SEQ ID NO: 5 as the nucleotide sequence of GATA2.

In an aspect, the protein-encoding sequence of GATA2 which includes the deletion is a pathogenic mutation. Exemplary mutations are frameshift mutations or premature termination mutations which can disrupt the entire protein sequence downstream of the mutation.

In another aspect, the deletion is an in-frame deletion of a GATA2 structural or functional motif.

In an aspect, the protein-encoding sequence of GATA2 which includes the deletion is a zinc finger-encoding region. In this aspect, the deletion can be an in-frame deletion of a zinc finger-encoding region. A zinc finger is a protein structural motif characterized by one or more coordinated zinc ions which stabilize the characteristic finger-like shape. In an aspect, the zinc-finger-encoding region comprises SEQ ID NO: 2 or SEQ ID NO: 3. In another aspect, the protein-encoding sequence of GATA2 which includes the in-frame deletion comprises two zinc finger-encoding regions, such as SEQ ID NO: 2 and SEQ ID NO: 3. In an aspect, the -frame deletion is represented by SEQ ID NO: 1.

In an aspect, the one copy of the GATA2 gene further comprises a deletion of a regulatory element of GATA2. In an aspect, the regulatory element of GATA2 is an intronic +9.5 enhancer. The intronic +9.5 enhancer has previously been shown to be essential for hematopoietic stem cell generation and stress hematopoiesis. Mutations in this enhancer are linked to GATA2-deficiency syndrome. Another regulatory element is 77,000 base pairs upstream of the murine Gata2 promoter (-77 enhancer), and this distal enhancer is conserved in humans but is located at -110 kb. This enhancer considerably elevates GATA2 expression in hematopoietic progenitor cells.

In an aspect, the deletion results in at least a 20%, 25%, 30%, 35%, 40%, 50%, 55%, 60%, 65%, 70% or higher reduction of GATA2 expression in the GATA2-deficient HUDEP cell line compared to a parent HUDEP cell line without the GATA2 deletion.

Also described herein is a method of preparing the GATA2-deficient HUDEP cell line described above, comprising introducing, e.g., transfecting, a Cas9 ribonucleoprotein comprising two guide RNAs into a population of HUDEP cells, wherein the Cas9 ribonucleoprotein cleaves GATA2 to provide the deletion of at least a portion of one or more protein-encoding sequences of GATA2, and propagating the GATA2-deficient HUDEP cell line. Propagating can include incubating the cells for a number of days on a plate and/or expanding the clones produced after introducing the Cas9 ribonucleoprotein. DNA sequencing can be used to verify the correct in-frame deletion has been produced.

CRISPR (Clustered Regularly Interspaced Short Palindromic Repeats) Cas9-mediated gene disruption has been widely used in generating loss-of-function mutations in diverse cell types including human cells. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog. In some embodiments, the CRISPR enzyme is codon-optimized for expression in a eukaryotic cell.

CRISPR/Cas9 is a ribonucleoprotein (RNP) complex. CRISPR RNA (crRNA) includes an about 20 base element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

Guide RNA, or gRNA, can be in the form of a crRNA/tracrRNA two guide system, or a single guide RNA. The guide RNA is capable of directing Cas9-mediated cleavage of target DNA. A guide RNA thus contains the sequences necessary for Cas9 binding and nuclease activity and a target sequence complementary to a target DNA of interest (protospacer sequence).

As used herein, a guide RNA protospacer binding sequence refers to the nucleotide sequence of a guide RNA that binds to a target genomic DNA sequence and directs Cas9 nuclease activity to a target DNA locus in GATA2. In some embodiments, the guide RNA protospacer binding sequence is complementary to the target DNA sequence. “Complementary” or “complementarity” refers to specific base pairing between nucleotides or nucleic acids. Base pairing between a guide RNA and a target region in GATA2, for example, can be via a DNA targeting sequence that is perfectly complementary or substantially complementary to the guide RNA. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the cleavage target includes a protospacer adjacent motif (PAM) adjacent to the protospacer region which is a sequence recognized by the CRISPR RNP. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyogenes has a NGG trinucleotide PAM motif; the PAM motif of N. meningitidis Cas9 is NNNNGATT (SEQ ID NO: 6); the PAM motif of S. thermophilus Cas9 is NNAGAAW(SEQ ID NO: 7); and the PAM motif of T. denticola Cas9 is NAAAAC (SEQ ID NO: 8).

A “Cas9” polypeptide is a polypeptide that functions as a nuclease when complexed to a guide RNA, e.g., an sgRNA or modified sgRNA. That is, Cas9 is an RNA-mediated nuclease. The Cas9 (CRISPR-associated 9, also known as Csn1) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

For example, the Cas9 may include, a Cas9 from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the variant may preferably increase specificity; for example, SpyFi Cas9 (Aldevron, Fargo, N. Dak.). In an exemplary embodiment, the Cas9 includes Streptococcus pyogenes Cas9 (SpCas9).

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

The term “cleavage site” refers to any site that can be cleaved by a CRISPR enzyme after binding to a target sequence. In general, wild type S. pyogenes Cas9 (SpCas9) is known to make a blunt cut between the 17th and 18th bases in the target sequence (3 bp 5′ of the PAM). As used herein the term “cleaves” generally refers to the generation of a double-stranded break in the DNA genome at a desired location.

The GATA2-deficient HUDEP cell lines described herein are particularly useful to screen mutations in GATA2. More than a hundred mutations to the protein coding sequence of GATA2 have been identified in patients with bone marrow failure and acute myeloid leukemia (AML). However, it is often unclear whether these mutations are neutral (benign) or negatively impact the function of GATA2 in such a way as to create a predisposition to develop bone marrow failure and/or leukemia (pathogenic). If pathogenic, how such mutations impact the function of GATA2 may not be predictable based on current knowledge. By expressing the GATA2 mutants in HUG2low cells, the impact of the mutation on GATA2 function can be evaluated by measuring changes to the transcriptome of these cells or any other parameter regulated by GATA2. This system represents a rapid and facile approach to distinguish GATA2 pathogenic from benign mutants and to elucidate GATA2 mechanisms in a human hematopoietic progenitor cell.

In an aspect, a method of screening a GATA2 mutation, comprises expressing a variant GATA2 comprising the GATA2 mutation in the GATA2-deficient HUDEP cell line as described herein, and determining if the variant GATA2 in the GATA2-deficient HUDEP cell line is pathogenic, likely pathogenic, or neutral.

In an aspect, the variant GATA2 is expressed in the GATA2-deficient HUDEP cell line with a lentiviral expression system. Lentiviral expression systems are well-known in the art and are commercially available from Addgene and Thermo Fischer Scientific, for example.

In an aspect, the variant GATA2 is a variant of uncertain significance (VUS). While many GATA2 variants have been shown to reduce GATA2 expression and/or function, many mutations identified in patients have unknown functional consequences. Identifying the activity and significance of these VUSs can elucidate the clinical significance of these variants and can impact clinical decision making for these patients.

As explained in detail in Bresnick et al. (“Human GATA2 mutations and hematologic disease: how many paths to pathogenesis”, Blood Advances, 4 (18), 4584-4592 (2020)), of the GATA2 mutations identified in ClinGen/ClinVar, a little less than half were designated as pathogenic, and more than half of the pathogenic variants were in the zinc finger domain. Pathogenic mutations outside of the zinc finger domain are often nonsense or frameshift mutations and are more likely to be VUSs.

Identified VUSs include V3A, A4E, E6K/Q, P8L/Q, W10C/R, H13Q, P14S, V16M, L17Q, Q20P, H21L/Q, P22R/S/T, S24L, H25L, H26N, M34T, P41S, V47G/I, F48I, F49L, N50S, S54A, N57T, Y59C, A61V, N62K, P63A/R/S, R69H, A75G/Y/V, H76R, G81A, P93Q, G100V, G101D, A104S/V, A109V, H113R, H113del, P120S, S122C, T124K/M, H127R, P128L, G133S, P137S, L138P, V140A, P142L, A144G, S148G, S152N/R, G153R, A157S, P161S, H165L/Q, S168A, P175S, P178S, E180A, V181M, T187S, A190S/T, A191T, S192F, A198T/V, G199V, G200D, E206Q, D209N, G210S, G210dup, V211L, K212N, M221L, M223I, S225N, P228T, R230C/H, M236L/T/V, P240L, A241V, T242A, H243Q/Y, P245S, T248I, P250A/S, Y252C, P254R, D259G, S262N, G263A/R, L264F, P267L, G268V, F270C, G272E, G273V, S277G, F278I, R283H, R287H, V382F, T387N, T387_K389del, K389_K390del, I393M, M400I, N402S, K406M, A41E/V, E412D, C413Y, F414L, E416D, M421I, S425L/P, P427R, F428I, S429N, A430V, A434P, A438S, P439S, V440M, P444L, G450R, P454S, P456L, T457M, P458S, I459N, S462Y, S464I, G468S, H469Q, P472L, AND M475L.

GATA2 mutations in children and adults can be asymptomatic, despite having family members with GATA2 deficiency syndrome. Somatic mutations in a host of genes (RUNX1, ETV6, CEBPA, ASXL1, SETBP1, and STAG2) occur commonly with germline GATA2 mutations and may constitute pathogenic triggers. However, whether different somatic mutation combinations combined with GATA2 germline mutation qualitatively or quantitatively influence pathogenesis is unknown.

In an aspect, the GATA2 mutation is a GATA2 missense mutation or a truncating mutation.

In an aspect, the GATA2 mutation is identified in a patient with GATA2 deficiency syndrome, chronic myelogenous leukemia (CML), or acute myeloid leukemia (AML). GATA2 deficiency syndrome involves immunodeficiency with monocytopenia; B cell, natural killer cell, and dendritic cell deficiencies; and common Mycobacterium, fungal, and viral infections. Patients with GATA2 deficiency syndrome may also exhibit lymphedema or monosomy 7. GATA2 mutations create a myelodysplastic syndrome/AML predisposition, and physiological GATA2 levels suppress bone marrow failure and leukemogenesis. The only potentially curative therapy for these conditions is bone marrow transplantation. CML is a cancer in which the bone marrow makes too many white blood cells. Typically, it affects older adults and is typically caused by spontaneous mutations. AML is a fast-growing cancer in which the bone marrow produces excess abnormal white blood cells. AML which is inherited is called familial AML.

In an aspect, the method, e.g., determining if the variant GATA2 in the GATA2-deficient HUDEP cell line is pathogenic, likely pathogenic, or neutral, comprises RNA sequencing and identifying differentially expressed genes for the variant GATA2 compared to wild-type GATA2.

In an aspect, the method, e.g., determining if the variant GATA2 in the GATA2-deficient HUDEP cell line is pathogenic, likely pathogenic, or neutral, comprises directly detecting DNA binding by the variant GATA2, although DNA binding may not be a reliable measure of activity.

In an aspect, the method, e.g., determining if the variant GATA2 in the GATA2-deficient HUDEP cell line is pathogenic, likely pathogenic, or neutral, comprises RNA sequencing and identifying differentially expressed genes for the variant GATA2 compared to wild-type GATA2. The activity of the variant GATA2 in the GATA2-deficient HUDEP cell line can be measured by detecting changes to the transcriptome of the GATA2-deficient HUDEPs.

The RNA-sequencing data can then be used in a gene ontology analysis to determine which biological activities are most impacted by the changes in expression for the variant GATA2. Gene ontology analysis can describe the functions of the variant GATA2, for example, the biological processes, cellular locations and/or molecular functions affected by the variant GATA2.

In an aspect, the differentially expressed genes comprise genes involved in signal transduction, such as MS4A2, PRG2 and/or RHEX.

In another aspect, the method, e.g., determining if the variant GATA2 in the GATA2-deficient HUDEP cell line is pathogenic, likely pathogenic, or neutral, comprises identifying gene regulatory regions bound by the variant GATA2 compared to wild-type GATA2 and/or chromatin occupancy of variant GATA2 compared to wild-type GATA.

GATA factors assemble and integrate into multiprotein complexes on chromatin that may enable tethering of a DNA binding–defective mutant into the complex. In another aspect, the activity of the variant GATA2 in the GATA2-deficient HUDEP cell line can be measured by a “Cut & Tag” procedure that permits selective recovery of chromatin fragments bound by transcriptional regulatory proteins or that possess particular histone modifications. Combined with global gene expression data obtained by RNA-sequencing, Cut & Tag can be used to identify important gene regulatory regions (e.g., enhancers) bound by GATA2. Furthermore, by comparing the patterns of chromatin occupancy of normal GATA2 with GATA2 amino acid variants, the contribution of variant amino acid sequences to chromatin occupancy can be determined.

Based on the determined activity to regulate the GATA2-dependant genetic network, the variant GATA2 can be characterized as pathogenic, likely pathogenic, or neutral (benign). Through the misregulation of components of the network, pathogenic variants can create a predisposition to develop bone marrow failure and/or leukemia, for example. In addition, the data can be used to elucidate GATA2-dependent pathogenic mechanisms by establishing whether variants impair GATA2 activity pre- or post-chromatin occupancy. If GATA2 variant chromatin occupancy is normal, Cut and Tag will be used to evaluate recruitment of co-regulatory proteins known to function in concert with GATA2. While Cut and Tag will be used to provide greater mechanistic insights, the pathogenic potential of GATA variants will be determined primarily by target gene expression.

Advantageously, using the methods described herein, the determination of the capacity of GATA2 mutants to rescue the transcriptomic defects can be ascertained in a few days, which is compatible with clinical decision-making. In this manner, several GATA2 mutations can be screened simultaneously. Ultimately, data generated using the cell lines described herein will provide clinicians with a metric for ascertaining the pathogenicity of the GATA2 mutations observed in patients and improve treatment options for these patients.

The invention is further illustrated by the following non-limiting examples.

### Examples

### Methods

To generate HUG2low cells, exons encoding zinc fingers and the +9.5 enhancer-containing intron were simultaneously deleted from one allele using custom synthetic gRNAs (Integrated DNA Technologies) targeting GCCGGGAGTGTGTCAACTGT (SEQ ID NO: 9) and AGACGACAACCACCACCTTA (SEQ ID NO: 10) in neighboring exons. sgRNA were assembled into ribonucleoprotein complexes with CAS9 protein (Integrated DNA Technologies, CAT #1081058) and introduced into HUDEP2 cells using a Lonza 4D Nucleofector® (P3 Buffer, program EO-100). Clones obtained by limiting dilution were sequence verified. Loss of GATA2 expression was confirmed by Western blotting. To rescue GATA2 deficiency, HA-tagged GATA2 was cloned into the plasmid pCDH-CMV-MCS-EF1and packaged into lentivirus in HEK293T cells using packaging plasmids psPAX2 (Addgene Plasmid #12260) and pMD2.G (Addgene Plasmid #12259). Virus-containing culture supernatant from the HEK293T cells was used to infect the HUG2low cells. Variants are introduced into GATA2 by site directed mutagenesis and sequence verified prior to packaging into lentivirus.

### Example 1: Generation and characterization of GATA2-deficient HUDEP-2 cells (HUG2low).

HUDEP-2 cells were developed in the laboratory of Yukio Nakamura (Kurita R, Suda N, Sudo K, Miharada K, Hiroyama T, Miyoshi H, Tani K, Nakamura Y: Establishment of immortalized human erythroid progenitor cell lines able to produce enucleated red blood cells. PLoS One 2013, 8:e59890.). To generate the HUDEP-2 cell line, a doxycycline-inducible HPV16-E6/E7 expression cassette was integrated into the genome of human CD34⁺ hematopoietic stem/progenitor cells isolated from umbilical cord blood. Consequently, HUDEP-2 cells proliferate indefinitely in cell culture as undifferentiated cells when grown in the presence of doxycycline. Removal of doxycycline and culture in media to support erythroid differentiation causes the cells to switch from a proliferative progenitor to a nonproliferative committed red cell fate. Proliferating HUDEP-2 cells express GATA2 and complete loss of GATA2 is likely lethal to HUDEP-2 cells as with other hematopoietic progenitors. However, a reduction in GATA2 protein levels can be tolerated though GATA2-deficient cells often exhibit pronounced changes in gene expression and have impaired differentiation potential. These characteristics were leveraged to study GATA2 function.

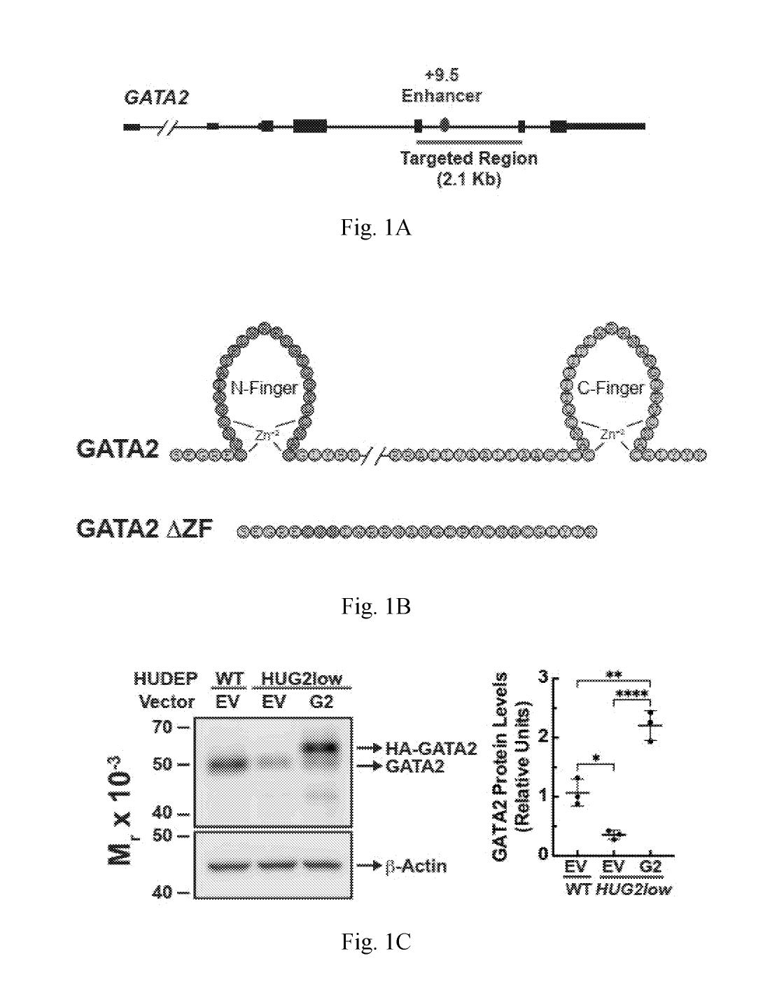

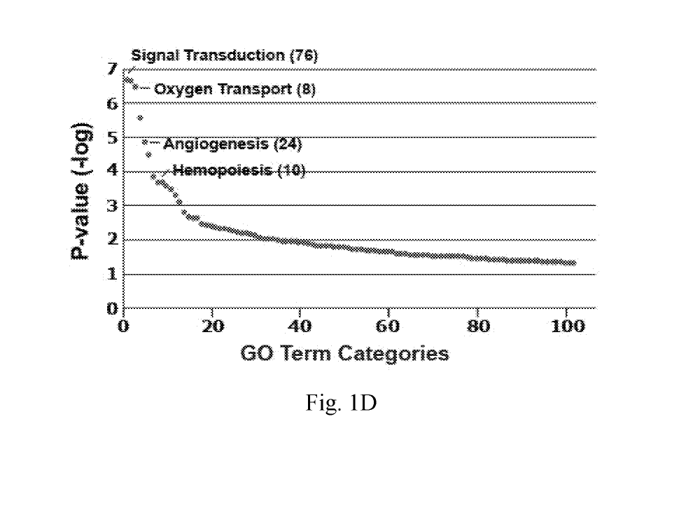

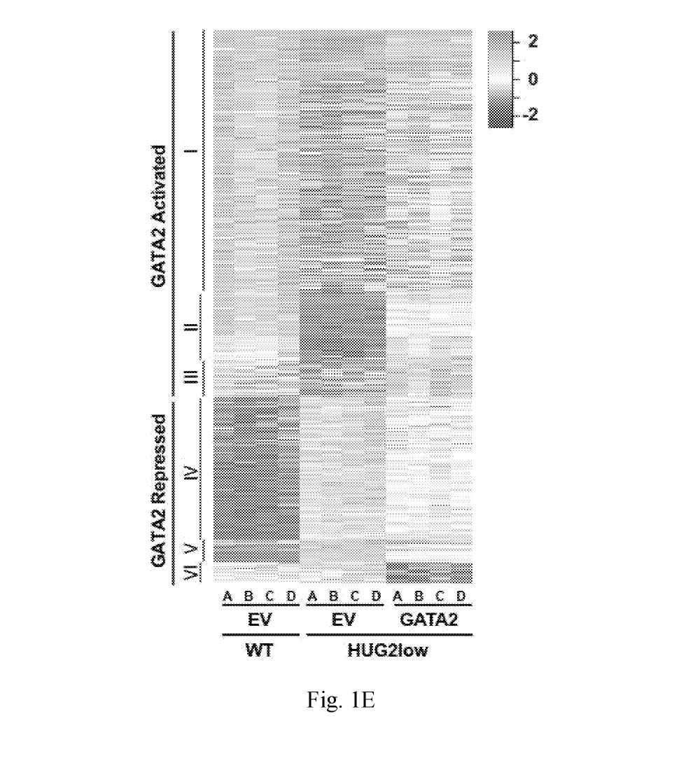

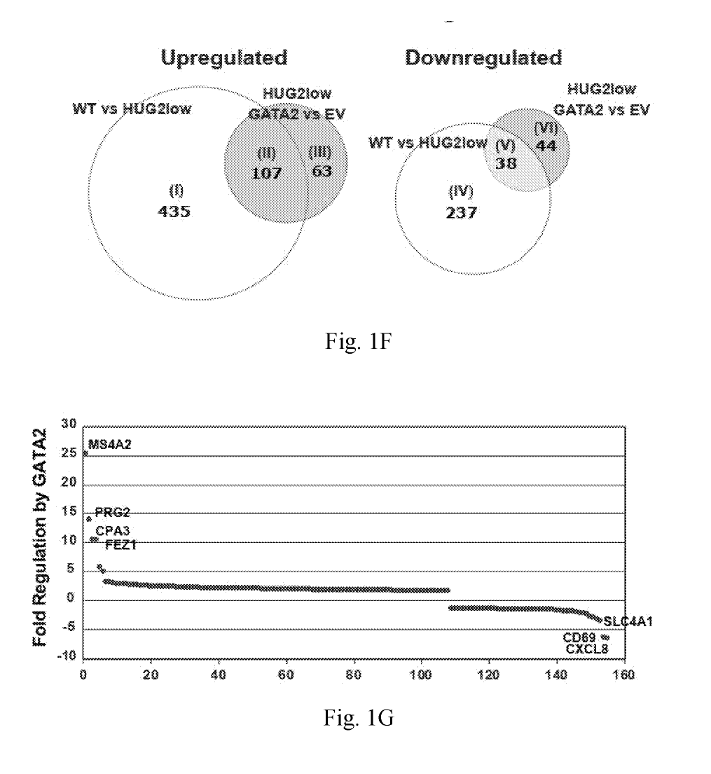

CRISPR-Cas9 methodology has been used to edit HUDEP-2 cells (Moir-Meyer et al., “Robust CRISPR-Cas9 Genome editing of the HUDEP-2 Erythroid Precursor Line Using Plasmids and Single-Stranded Oligonucleotide Donors”, Methods and Protoc., 2018, 1, 28; doi:10.3390/mps1030028). CRISPR-CAS editing of cells including HUDEP-2 cells is also described in US2024/0102004, incorporated herein by referencing for this teaching. A GATA2-deficient HUDEP-2 cell line was prepared using CRISPR-Cas9 methodology to delete 2.1 kb from one allele of GATA-2 (FIG. 1A). This was accomplished using two synthetic sgRNAs (IDT) assembled into a ribonucleoprotein complex with Cas9 protein and introduced into HUDEP-2 cells using a Lonza 4-D Nucleofector®. The resulting in-frame deletion removed DNA sequence encoding most of the two zinc fingers of GATA2 that mediate DNA binding and protein-protein interactions (FIG. 1B). Also deleted was the intronic +9.5 enhancer previously shown to be essential for hematopoietic stem cell generation and stress hematopoiesis. Mutations in this enhancer are linked to GATA2-deficiency syndrome. Quantitation of protein levels by Western blotting showed that the targeted cells produce approximately 66% less GATA2 (FIG. 1C). These cells are termed HUDEP-2 GATA2 low cells (HUG2low). A quantitative RNA-sequencing comparison of the transcriptomes of wild-type HUDEP-2 cells and HUG2low cells revealed that substantial changes in gene expression were induced by GATA2 deficiency with 828 genes differentially expressed ≥1.5-fold between the two cell lines. Gene ontology analysis revealed changes in the expression of cohorts of genes involved in signal transduction, oxygen transport, angiogenesis and hemopoiesis to name a few (FIG. 1D). Genes found to be differentially expressed between HUDEP-2 and HUG2low cells can be classified as GATA2 activated (decreased levels in HUG2low cells) or GATA2 repressed (increased levels in HUG2low cells). Rescue of the gene expression defects was evaluated in the HUG2low cells by ectopic expression of GATA2 possessing an HA-tag (HA-GATA2, FIG. 1C). Doing so, the GATA2 activated and repressed genes were classified based on responsiveness to rescue as being nonresponsive (groups I and IV) or responsive (groups II and V). Genes that were not affected by loss of GATA2 in HUG2low cells but that were uniquely responsive to expression of HA-GATA2 were identified (groups III and VI) (FIG. 1E, and F). The magnitude of change in gene expression for most the 145 genes of groups II and V was 1.5-3.0-fold, comparable to the change in GATA2 levels though some genes (e.g., MS4A2, PRG2, CAP3, FEZ1, CXCL8 and CD69) proved to be hypersensitive, changing in expression by as much as 25-fold when HA-GATA2 was introduced into the cells (FIG. 1G).

### Example 2: Evaluation of GATA2 variants

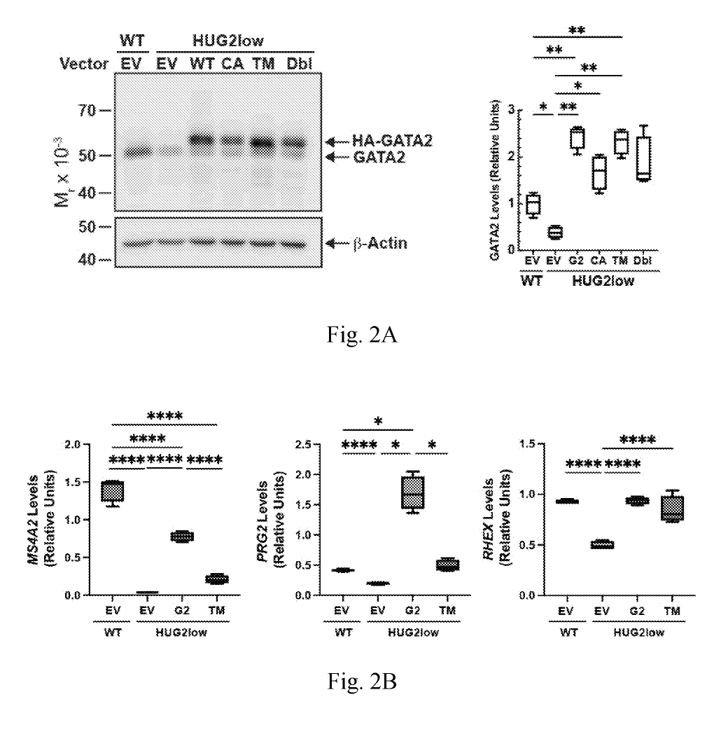

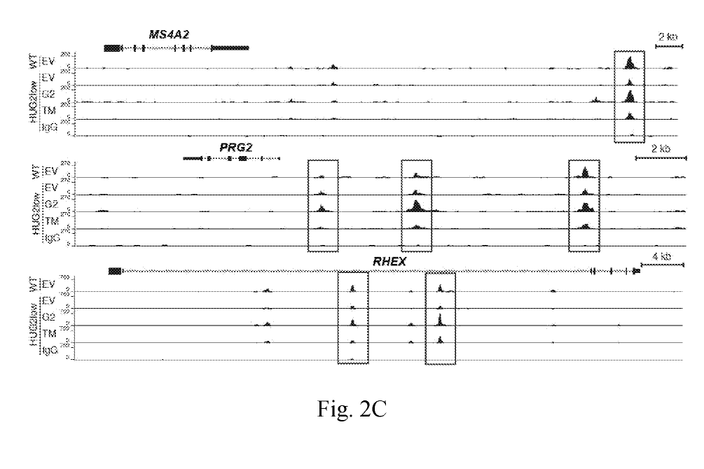

The identification of a cohort of genes in HUG2low cells that are responsive to ectopic GATA2 expression provides a system by which the transcriptional activity of GATA2 variants can be evaluated. Using a lentiviral-based expression system, wild-type GATA2 and VUSs can be expressed in HUG2low cells with high efficiency (80-100%). The inclusion of an HA-tag enables the expressed GATA2 to be distinguished from endogenous GATA2 (FIG. 1C). GATA2 functions as a DNA binding transcriptional regulator. Amino acid sequences within GATA2 utilize zinc atoms to assemble into three-dimensional structures termed zinc-finger domains that mediate interactions with DNA and other proteins. Amino acid variants may disrupt zinc finger assembly or alter affinity for DNA or other proteins leading to reduced GATA2 stability or activity. Pathogenic variants have been identified at amino acids residing in unstructured regions of GATA2 outside of the zinc finger domains, indicating they also contribute to GATA2 activities. If the screens identify variants defective at rescuing GATA2 target gene expression, these variants will be further evaluated for protein stability, intracellular localization, DNA binding activity and interactions with other transcriptional regulatory proteins. To demonstrate that HUG2low cells can be used to segregate the activity of a GATA2 variant from wild-type, a known GATA2 pathogenic variant was expressed in the cells. The T354M variant, a threonine to methionine substitution at amino acid 354 located in one of the zinc-fingers, is known to disrupt DNA binding. When expressed in HUG2low cells at levels comparable to wild-type (FIG. 2A), the T354M variant showed reduced ability to activate the expression of MS4A2 and PRG2 but could activate RHEX (FIG. 2B). Using the Cut & Tag approach to quantitate DNA binding genome-wide, binding of endogenous GATA2 was reduced in HUG2low cells compared to HUDEP-2 cells (FIG. 2C). Though wild-type HA-GATA2 restored DNA binding to near normal levels, the TM variant had little, if any, effect at most genes (e.g., MS4A2 and PRG2) but partially rescued occupancy at others (e.g., RHEX). These studies demonstrate the importance of evaluating multiple parameters as GATA2 variants may selectively impact a subset of GATA2 targets rather than the full ensemble.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A GATA2-deficient human umbilical cord blood-derived erythroid progenitor (HUDEP) cell line, wherein one GATA2 gene copy comprises a deletion of at least a portion of one or more GATA2 protein-encoding sequences, wherein the deletion results in at least a 25% reduction of GATA2 expression in the GATA2-deficient HUDEP cell line compared to a parent HUDEP cell line without the GATA2 deletion.

2. The GATA2-deficient HUDEP cell line of claim 1, wherein the HUDEP cell line is HUDEP-2.

3. The GATA2-deficient HUDEP cell line of claim 1, wherein the deletion is a frameshift mutation or a premature termination mutation.

4. The GATA2-deficient HUDEP cell line of claim 1, wherein the one or more protein-encoding sequences of GATA2 comprises a zinc-finger-encoding region, and wherein the deletion is an in-frame deletion.

5. The GATA2-deficient HUDEP cell line of claim 4, wherein the zinc-finger-encoding region has the sequence of SEQ ID NO: 2 or SEQ ID NO: 3.

6. The GATA2-deficient HUDEP cell line of claim 1, wherein one or more protein-encoding sequences of GATA2 comprises two zinc finger encoding regions, and wherein the deletion is an in-frame deletion.

7. The GATA2-deficient HUDEP cell line of claim 6, wherein the two zinc-finger encoding regions have the sequences of SEQ ID NO: 2 and SEQ ID NO: 3.

8. The GATA2-deficient HUDEP cell line of claim 7, wherein the deletion has the sequence of SEQ ID NO: 1.

9. The GATA2-deficient HUDEP cell line of claim 8, wherein the HUDEP cell line is HUDEP-2.

10. The GATA2-deficient HUDEP cell line of claim 1, wherein the one copy of the GATA2 gene further comprises a deletion of a regulatory element of GATA2.

11. The GATA2-deficient HUDEP cell line of claim 10, wherein the regulatory element of GATA2 is an intronic +9.5 enhancer.

12. A method of screening a GATA2 mutation, comprising
expressing a variant GATA2 comprising the GATA2 mutation in the GATA2-deficient HUDEP cell line of claim 1, and
determining if the variant GATA2 in the GATA2-deficient HUDEP cell line is pathogenic, likely pathogenic, or neutral.

13. The method of claim 12, wherein the variant GATA2 is expressed in the GATA2-deficient HUDEP cell line with a lentiviral expression system.

14. The method of claim 12, wherein the variant GATA2 is a variant of uncertain significance.

15. The method of claim 12, wherein the GATA2 mutation is a GATA 2 missense mutation or a truncating mutation.

16. The method of claim 12, wherein the GATA2 mutation is identified in a patient with GATA2 deficiency syndrome, chronic myelogenous leukemia, or acute myeloid leukemia.

17. The method of claim 12, wherein the determining comprises RNA sequencing and identifying differentially expressed genes for the variant GATA2 compared to wild-type GATA2.

18. The method of claim 17, further comprising performing gene ontology analysis using RNA sequencing data to determine biological activities impacted by the variant GATA2 compared to wild-type GATA2.

19. The method of claim 17, wherein the differentially expressed genes are involved in signal transduction for the variant GATA2 compared to wild-type GATA2.

20. The method of claim 19, wherein the genes involved in signal transduction comprise MS4A2 PRG2 and/or RHEX.

21. The method of claim 12, wherein determining comprises identifying gene regulatory regions bound by the variant GATA2 compared to wild-type GATA2 and/or chromatin occupancy of variant GATA2 compared to wild-type GATA2.
